(19) United States
(12) Patent Application Publication
(10) Pub. No.: US 2026/0137672 A1
(43) Pub. Date: May 21, 2026

(54) SUSTAINED-RELEASE TABLET COMPRISING APIXABAN

(71) Applicant: Rexpharmtech Co., Ltd, Yongin-si, Gyeonggi-do (KR)

(72) Inventors: Ji Seok YOO, Suwon-si, Gyeonggi-do (KR); Yongnam LEE, Yongin-si, Gyeonggi-do (KR); Dae Hyeon KIM, Yongin-si, Gyeonggi-do (KR); Hye Jeong LEE, Yongin-si, Gyeonggi-do (KR)

(73) Assignee: Rexpharmtech Co., Ltd, Yongin-si, Gyeonggi-do (KR)

(21) Appl. No.: 19/122,411

(22) PCT Filed: Dec. 26, 2023
(86) PCT No.: PCT/KR2023/021585

§ 371 Date: Apr. 18, 2025

(30) Foreign Application Priority Data
Dec. 26, 2022 (KR) 10-2022-0184502

(51) Int. Cl. A61K 31/4545; A61K 9/20

(52) U.S. Cl. CPC A61K 31/4545; A61K 9/2013; A61K 9/2031

(57) ABSTRACT

Provided is a once-daily sustained-release tablet containing apixaban, the tablet comprising apixaban or a pharmaceutically acceptable salt thereof, polyethylene oxide as a sustained-release base and tocopherol polyethylene glycol succinate.

### TECHNICAL FIELD

The present disclosure relates to a sustained-release tablet comprising apixaban or a pharmaceutically acceptable salt thereof, polyethylene oxide as a sustained-release base, and tocopherol polyethylene glycol succinate.

### BACKGROUND ART

The chemical name for apixaban is 4,5,6,7-tetrahydro-1-(4-methoxyphenyl)-7-oxo-6-[4-(2-oxo-1-piperidinyl)phenyl]-1H-pyrazolo[3,4-c]pyridine-3-carboxamide [Chemical Abstract Service (CAS) name] or 1-(4-methoxyphenyl)-7-oxo-6-[4-(2-oxopiperidin-1-yl)phenyl]-4,5,6,7-tetrahydro-1H-pyrazolo[3,4-c]pyridine-3-carboxamide.

Apixaban is a factor Xa inhibitor disclosed in U.S. Pat. No. 6,967,208 and is an antithrombotic agent for the purpose of preventing venous thromboembolism, treating deep venous thrombosis (DVT) and pulmonary embolism (PE), and reducing the risk of recurrence of DVT and PE. In Korea, it is sold as 2.5 mg and 5 mg tablets under the brand name Eliquis. Apixaban tablets currently on the market are sold only in the form of administration twice a day, depending on the half-life of the drug.

Patients receiving apixaban have the inconvenience of having to take the drug once in the morning and once in the evening according to the approved dosage of twice a day, and also have the inconvenience of having to take the medication immediately and again every 12 hours if they forget to take the medicine.

Meanwhile, the development of apixaban from the previously approved twice-daily formulation into a once-daily formulation requires technology for making sustained-release preparation. However, apixaban has a problem in achieving a predetermined bioavailability depending on the dosage form (Sci Pharm. 2014(82)777-785).

### DISCLOSURE OF INVENTION

### Technical Problem

An object of the present disclosure is to provide a sustained-release tablet comprising apixaban or a pharmaceutically acceptable salt thereof, polyethylene oxide as a sustained-release base, and tocopherol polyethylene glycol succinate.

### Solution to Problem

The present disclosure will be described in detail as follows. Meanwhile, each description and embodiment disclosed in the present disclosure may be applied to each of the other descriptions and embodiments. In other words, all combinations of various elements disclosed in the present disclosure fall within the scope of the present disclosure. In addition, it cannot be considered that the scope of the present disclosure is limited by specific descriptions described below.

The present disclosure relates to a sustained-release tablet comprising apixaban or a pharmaceutically acceptable salt thereof, polyethylene oxide as a sustained-release base, and tocopherol polyethylene glycol succinate.

In the present disclosure, apixaban refers to a compound having the structure of the following Chemical Formula 1:

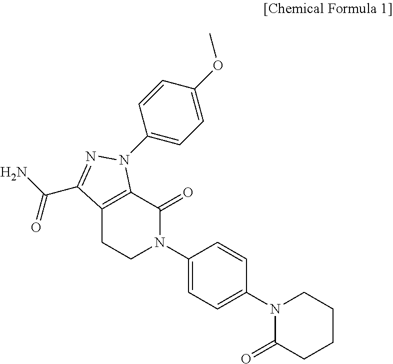 [SMILES: COc1ccc(-n2nc(C(N)=O)c3c2C(=O)N(c2ccc(N4CCCCC4=O)cc2)CC3)cc1]

Apixaban is an oral anticoagulant used to inhibit blood clotting. Apixaban reduces the risk of embolism, in which blood vessels are blocked by blood clots, in diseases prone to thrombosis, such as non-valvular atrial fibrillation and deep venous thrombosis after hip or knee replacement surgery.

In the sustained-release tablet of the present disclosure, apixaban may be in free base or salt form, solvate, or anhydrous form.

The apixaban of the present disclosure may be present in the form of a “pharmaceutically acceptable salt”. As the salt, an acid addition salt formed by a pharmaceutically acceptable free acid is useful. As used herein, the term “pharmaceutically acceptable salt” refers to any organic or inorganic addition salt of the compound of which concentration has effective action that is relatively non-toxic and harmless to patients in which side effects caused by these salts do not degrade the beneficial efficacy of apixaban.

The acid addition salts are prepared by conventional methods, for example, by dissolving a compound in an excess aqueous acid solution, and precipitating the salt using a water-miscible organic solvent, such as methanol, ethanol, acetone or acetonitrile. Here, an equimolar amount of the compound and an acid or alcohol in water may be heated, and then the mixture may be evaporated to dryness, or the precipitated salt may be suction filtered.

During the process, as the free acid, organic acids and inorganic acids may be used, in which the inorganic acid may include hydrochloric acid, phosphoric acid, sulfuric acid, or nitric acid, and the like, and the organic acid may include methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, or hydroiodic acid, and the like. However, the organic acid and inorganic acid are not limited thereto.

The sustained-release tablet of the present disclosure comprises polyethylene oxide as a sustained-release base. Polyethylene oxide suitable for use in the present disclosure is commercially available, for example, under the brand name POLYOX from Dow Chemical Company having a molecular weight of 100,000 to 7,000,000.

In the sustained-release tablet of the present disclosure, apixaban or a pharmaceutically acceptable salt thereof may be contained in an amount of 2 to 3% by weight based on the total weight of a uncoated tablet.

Preferably, the sustained-release tablet of the present disclosure may comprise apixaban in an amount of 10 mg.

Preferably, the sustained-release tablet of the present disclosure may comprise the polyethylene oxide in an amount of 30% to 60% by weight based on the total weight of the uncoated tablet.

When the amount of polyethylene oxide is less than 30% by weight or more than 60% by weight based on the total weight of the uncoated sustained-release tablet, it is not possible to achieve sufficient sustained release of apixaban, or sustained release may excessively occur, making it impossible to exhibit a desirable dissolution pattern as a sustained release preparation for once-daily administration. Eventually, there is a problem of causing biological non-equivalence in animal pharmacokinetic tests with the reference drug (for example, Eliquis tablets).

Preferably, the sustained-release tablet of the present disclosure may comprise the polyethylene oxide in an amount of 37.5% to 60% by weight based on the total weight of the uncoated tablet.

Most preferably, the sustained-release tablet of the present disclosure may comprise the polyethylene oxide in an amount of 37.5% to 45% by weight based on the total weight of the uncoated tablet.

In addition, preferably, the polyethylene oxide of the present disclosure may be at least any one selected from the group consisting of polyethylene oxide 300,000 and polyethylene oxide 1,000,000.

Preferably, the polyethylene oxide contained as the sustained-release base in the present disclosure may be polyethylene oxide 300,000, polyethylene oxide 1,000,000, or a mixture of polyethylene oxide 300,000 and polyethylene oxide 1,000,000.

In addition, preferably, in the sustained-release tablet of the present disclosure, the sum of weights of the polyethylene oxide 300,000 and the polyethylene oxide 1,000,000 may be 40% by weight to 60% by weight based on the total weight of the uncoated tablet.

In addition, more preferably, in the sustained-release tablet of the present disclosure, the sum of weights of the polyethylene oxide 300,000 and the polyethylene oxide 1,000,000 may be 40% by weight to 50% by weight based on the total weight of the uncoated tablet.

In the present disclosure, when the mixture of polyethylene oxide 300,000 and polyethylene oxide 1,000,000 is used as the sustained-release base and the sum of weights of polyethylene oxide 300,000 and polyethylene oxide 1,000,000 in the sustained-release tablet is less than 40% by weight or more than 60% by weight based on the total weight of the uncoated tablet, there are problems in that the initial dissolution rate of the formulation decreases and biological non-equivalence occurs in animal pharmacokinetic tests with the reference drug (Eliquis tablets).

Preferably, the weight ratio of the polyethylene oxide 300,000 and the polyethylene oxide 1,000,000 in the present disclosure may be 1:0.5 to 1:3.

In addition, more preferably, the weight ratio of the polyethylene oxide 300,000 and the polyethylene oxide 1,000,000 in the present disclosure may be 1:1 to 1:2.

The sustained-release tablet of the present disclosure comprises tocopherol polyethylene glycol succinate as a lipophilic surfactant.

In an experimental example of the present disclosure, the present inventors prepared a sustained-release tablet containing apixaban, polyethylene oxide, and tocopherol polyethylene glycol succinate, and a sustained-release tablet containing apixaban and polyethylene oxide, but not containing tocopherol polyethylene glycol succinate or sustained-release tablets containing apixaban, polyethylene oxide, and different types of surfactants, and performed comparative dissolution tests and PK tests. As a result, it was confirmed that, the tablets without containing tocopherol polyethylene glycol succinate or containing the different types of surfactants showed significantly high initial dissolution rate (1-3 hours after dissolution), but the PK test results thereof showed that the AUC was significantly lower than that of the reference drug (Eliquis 5 mg tablet), making them unsuitable for once-daily sustained-release formulation.

In the sustained-release tablet of the present disclosure, the tocopherol polyethylene glycol succinate may be contained in an amount of 0.25% by weight to 2.5% by weight based on the total weight of the uncoated sustained-release tablet.

In the present disclosure, when the tocopherol polyethylene glycol succinate is contained in an amount exceeding 2.5% by weight based on the total weight of the uncoated sustained-release tablet, there may be a problem in that it is not possible to prepare tablets due to poor compression moldability.

In addition, in the present disclosure, when the tocopherol polyethylene glycol succinate is not contained in the sustained-release tablet or is contained in less than 0.25% by weight based on the total weight of the uncoated sustained-release tablet, the dissolution rate of apixaban may be rather increased, making it unsuitable for use as a once-daily sustained-release tablet.

More preferably, the tocopherol polyethylene glycol succinate of the present disclosure may be contained in an amount of 0.75% by weight to 2.5% by weight based on the total weight of the uncoated sustained-release tablet.

Most preferably, the tocopherol polyethylene glycol succinate of the present disclosure may be contained in an amount of 0.75% by weight to 1.25% by weight based on the total weight of the uncoated sustained-release tablet.

The sustained-release tablet of the present disclosure may further comprise pharmaceutically acceptable additives, if necessary, selected from the group consisting of, for example, excipients, solubilizers, antioxidants, binders, lubricants, coating agents, and any combinations thereof.

Specifically, the sustained-release tablet of the present disclosure may comprise an excipient, a solubilizer, an antioxidant, a binder, a lubricant, and a coating agent.

The excipient may be any excipient known in the art, and for example, may be selected from the group consisting of lactose, anhydrous lactose, microcrystalline cellulose, mannitol, low-substituted hydroxypropylcellulose, calcium phosphate, light anhydrous silicic acid, colloidal silicon dioxide, magnesium aluminometasilicate, pregelatinized starch, corn starch, potato starch, white sugar, dextrin, precipitated calcium carbonate, and any combination thereof, but is not limited thereto.

The solubilizer may be any solubilizer known in the art, and for example, may be selected from the group consisting of sodium lauryl sulfate, sodium stearate, polysorbate, poloxamer, and any combination thereof, but is not limited thereto.

The antioxidant may be any antioxidant known in the art, and may be selected from the group consisting of butylated hydroxytoluene (BHT), butylated hydroxyanisole (BHA), and any combination thereof, but is not limited thereto.

The binder may be any binder known in the art. Preferably, the binder may be at least any one selected from the group consisting of low-substituted hydroxypropylcellulose, polyvinylpyrrolidone, povidone, and copovidone.

The lubricant may be any lubricant known in the art, and for example, may be selected from the group consisting of stearic acid, magnesium stearate, silicon dioxide, talc, sucrose fatty acid ester, hydrogenated vegetable oil, high melting point wax, glyceryl fatty acid esters, glycerol dibehenate, and any combination thereof, but is not limited thereto.

The coating agent may contain hydroxypropyl methylcellulose, ethylcellulose, polyvinylacetate, polyethylene glycol, titanium dioxide, iron oxide, and the like, or the brand name Opadry®.

The sustained-release tablet of the present disclosure may be a general tablet or may be contained in a capsule in the form of a mini-tablet, and if necessary, may be a film-coated tablet coated with a film.

Preferably, in the sustained-release tablet of the present disclosure, the apixaban may be contained in an amount of 2 to 3% by weight based on the total weight of a uncoated tablet, the excipient may be contained in an amount of 30 to 55% by weight based on the total weight of the uncoated tablet, the solubilizer may be contained in an amount of 0.1 to 2% by weight based on the total weight of the uncoated tablet, the antioxidant may be contained in an amount of 0.1% to 1% by weight based on the total weight of the uncoated tablet, the binder may be contained in an amount of 0.1 to 2% by weight based on the total weight of the uncoated tablet, and the lubricant may be contained in an amount of 0.1 to 2% by weight based on the total weight of the uncoated tablet.

Preferably, in the sustained-release tablet of the present disclosure, the apixaban may be contained in an amount of 2 to 3% by weight based on the total weight of the uncoated tablet, the excipient may be contained in an amount of 30 to 55% by weight based on the total weight of the uncoated tablet, the solubilizer may be contained in an amount of 0.1 to 2% by weight based on the total weight of the uncoated tablet, the antioxidant may be contained in an amount of 0.1% to 1% by weight based on the total weight of the uncoated tablet, the binder may be contained in an amount of 0.1 to 2% by weight based on the total weight of the uncoated tablet, the lubricant may be contained in an amount of 0.1 to 2% by weight based on the total weight of the uncoated tablet, the polyethylene oxide may be contained in an amount of 37.5% to 60% by weight based on the total weight of the uncoated tablet, and the tocopherol polyethylene glycol succinate may be contained in an amount of 0.25% to 2.5% by weight based on the total weight of the uncoated tablet.

Preferably, in the sustained-release tablet of the present disclosure, the apixaban may be contained in an amount of 2 to 3% by weight based on the total weight of the uncoated tablet, the excipient may be contained in an amount of 45 to 55% by weight based on the total weight of the uncoated tablet, the solubilizer may be contained in an amount of 0.1 to 1% by weight based on the total weight of the uncoated tablet, the antioxidant may be contained in an amount of 0.1% to 1% by weight based on the total weight of the uncoated tablet, the binder may be contained in an amount of 0.1 to 2% by weight based on the total weight of the uncoated tablet, the lubricant may be contained in an amount of 0.1 to 2% by weight based on the total weight of the uncoated tablet, the polyethylene oxide may be contained in an amount of 37.5% to 45% by weight based on the total weight of the uncoated tablet, and the tocopherol polyethylene glycol succinate may be contained in an amount of 0.75% to 2.5% by weight based on the total weight of the uncoated tablet.

Preferably, the sustained-release tablet of the present disclosure is apixaban tablet for once-daily administration.

Preferably, the sustained-release tablet of the present disclosure shows a dissolution rate of apixaban of 20% or less at 60 minutes, 40 to 60% at 180 minutes, and 80% or more at 360 minutes in a dissolution test with a pH 6.8 buffer solution at 100 rpm and according to the dissolution test method 2 of the Korean Pharmacopoeia General Test Method.

Further, the present disclosure provides a method for preparing apixaban sustained-release tablets, comprising:
Step 1 of preparing a binder solution by dissolving tocopherol polyethylene glycol succinate, a binder, and an antioxidant in a solvent;
Step 2 of preparing granules by employing a mixture of apixaban or a pharmaceutically acceptable salt thereof, polyethylene oxide as a sustained-release base, and a solubilizer using the binder solution of Step 1 above;
Step 3 of drying and milling the granules of Step 2 to obtain a mixture; and
Step 4 of tableting after mixing an excipient and a lubricant with the mixture of Step 3.

Details of the preparation method of the apixaban sustained-release tablet may be directly applied to the description of the sustained-release tablet of the present disclosure. Specifically, the apixaban, the pharmaceutically acceptable salt thereof, the polyethylene oxide, the tocopherol polyethylene glycol succinate, the binder, the antioxidant, the solubilizer, the excipient, and the lubricant are the same as described above.

The solvent used in the binder solution in the Step 1 may be, for example, ethanol, but is not limited thereto.

The preparation method of the present disclosure may further comprise, after the Step 4, a step of coating the tablets, if necessary.

The preparing of the granules, the drying and milling of the granules, and tableting of the obtained mixture used in the method for preparing the sustained-release tablets of the present disclosure may be performed by any conventional means and methods known in the art.

### Advantageous Effects of Invention

The sustained-release tablet of the present disclosure has an appropriate dissolution rate and is bioequivalent to Eliquis tablet, ensuring safety and effectiveness, and enabling to be administered once a day by improving the inconvenience of conventional twice-daily formulation.

### BRIEF DESCRIPTION OF DRAWINGS

FIGS. 1 and 2 show the results of dissolution tests at pH 6.8 in Examples 1 to 4 and Comparative Examples 1 to 7.

FIG. 3 shows the results of beagle dog pharmacokinetics tests using Example 2, Comparative Examples 3 and 6, and Eliquis 5 mg tablet as a reference drug.

### BEST MODE FOR CARRYING OUT THE INVENTION

Hereinafter, the constitution and effects of the present disclosure will be described in more detail through the following Examples. These Examples are only provided for illustrating the present disclosure, but the scope of the present disclosure is not limited by these Examples.

### Examples 1 to 4 and Comparative Examples 1 to 7: Preparation of Apixaban Tablets

Pharmaceutical compositions containing apixaban according to Examples 1 to 4 and Comparative Examples 1 to 7 were prepared using the ingredients and amounts in Table 1 below.

Specifically, a binder solution was prepared by dissolving tocopherol polyethylene glycol succinate (TPGS), propylene glycol monocaprylate (Capriol 90), glycerol monooleate or coco betaine, low-substituted hydroxypropylcellulose (L-HPC) and butylated hydroxy toluene (BHT) in 95% ethanol, and then granules were prepared by granulating a mixture of apixaban, polyethylene oxide (PEO) 1,000,000, polyethylene oxide (PEO) 300,000, anhydrous lactose, and sodium lauryl sulfate using the binder solution. The granules were subjected to drying and milling, and then anhydrous lactose, magnesium stearate, and silicon dioxide were added thereto and mixed with each other. The obtained mixture was tableted and coated to prepare apixaban tablets.

TABLE 1

| | | Example | Example | Example | Example | Comparative | Comparative |
|---|---|---|---|---|---|---|---|
| | | 1 | 2 | 3 | 4 | Example 1 | Example 2 |
| Classification | Component name | Amount (Unit: mg) | | | | | |
| Mixing and | Apixaban | 10.0 | 10.0 | 10.0 | 10.0 | 10.0 | 10.0 |
| Granulating | Anhydrous lactose | 88.6 | 88.6 | 118.6 | 118.6 | 88.6 | 88.6 |
| | PEO 1,000,000 | 80.0 | 60.0 | 0.0 | 150.0 | 80.0 | 0.0 |
| | PEO 300,000 | 80.0 | 120.0 | 150.0 | 0.0 | 0.0 | 80.0 |
| | Sodium lauryl sulfate | 1.0 | 1.0 | 1.0 | 1.0 | 1.0 | 1.0 |
| Binder | TPGS | 3.0 | 5.0 | 10.0 | 10.0 | 5.0 | 5.0 |
| solution | Propylene glycol monocaprylate | — | — | — | — | — | — |
| | Glycerol monooleate | — | — | — | — | — | — |
| | Coco betaine | — | — | — | — | — | — |
| | Ethanol | 83.2 | 83.2 | 83.2 | 83.2 | 83.2 | 83.2 |
| | BHT | 0.4 | 0.4 | 0.4 | 0.4 | 0.4 | 0.4 |
| | L-HPC | 5.0 | 5.0 | 5.0 | 5.0 | 5.0 | 5.0 |
| Post-mixing | Anhydrous lactose | 127.0 | 105.0 | 100.0 | 100.0 | 205.0 | 205.0 |
| | Magnesium stearate | 2.5 | 2.5 | 2.5 | 2.5 | 2.5 | 2.5 |
| | Silicon dioxide | 2.5 | 2.5 | 2.5 | 2.5 | 2.5 | 2.5 |
| Uncoated Tablet | | 400.0 | 400.0 | 400.0 | 400.0 | 400.0 | 400.0 |
| Coating agent | Colorcon Opadry 85F18422 | 12.0 | 12.0 | 12.0 | 12.0 | 12.0 | 12.0 |
| Final product | | 412.0 | 412.0 | 412.0 | 412.0 | 412.0 | 412.0 |

| | | Comparative | Comparative | Comparative | Comparative | Comparative |
|---|---|---|---|---|---|---|
| | | Example 3 | Example 4 | Example 5 | Example 6 | Example 7 |
| Classification | Component name | Amount (Unit: mg) | | | | |
| Mixing and | Apixaban | 10.0 | 10.0 | 10.0 | 10.0 | 10.0 |
| Granulating | Anhydrous lactose | 88.6 | 93.6 | 88.6 | 88.6 | 88.6 |
| | PEO 1,000,000 | 60.0 | 60.0 | 80.0 | 80.0 | 80.0 |
| | PEO 300,000 | 120.0 | 120.0 | 80.0 | 80.0 | 80.0 |
| | Sodium lauryl sulfate | 1.0 | 1.0 | 1.0 | 1.0 | 1.0 |
| Binder | TPGS | — | — | — | — | — |
| solution | Propylene glycol monocaprylate | — | — | 3.0 | — | — |
| | Glycerol monooleate | — | — | — | 3.0 | — |
| | Coco betaine | — | — | — | — | 3.0 |
| | Ethanol | 83.2 | 83.2 | 83.2 | 83.2 | 83.2 |
| | BHT | 0.4 | 0.4 | 0.4 | 0.4 | 0.4 |
| | L-HPC | 5.0 | 5.0 | 5.0 | 5.0 | 5.0 |
| Post-mixing | Anhydrous lactose | 110.0 | 110.0 | 127.0 | 127.0 | 127.0 |
| | Magnesium stearate | 2.5 | 2.5 | 2.5 | 2.5 | 2.5 |
| | Silicon dioxide | 2.5 | 2.5 | 2.5 | 2.5 | 2.5 |
| Uncoated Tablet | | 400.0 | 400.0 | 400.0 | 400.0 | 400.0 |
| Coating agent | Colorcon Opadry 85F18422 | 12.0 | 12.0 | 12.0 | 12.0 | 12.0 |
| Final product | | 412.0 | 412.0 | 412.0 | 412.0 | 412.0 |

### Experimental Example 1: Dissolution Test

Dissolution tests were performed on the preparations of Examples 1 to 4 and Comparative Examples 1 to 7. The dissolution test was performed with a pH 6.8 buffer solution at 100 rpm according to the dissolution test method 2 of the Korean Pharmacopoeia General Test Method.

### <HPLC Analysis Conditions>

Detector: 280 nm

Column: C18 5 cm 4.6 mm×150 mm

Column temperature: 35° C.

Sample temperature: 25° C.

Injection amount: 50 μL

Flow rate: 1.0 mL/min

Mobile phase: 10 mM (0.77 g/L) ammonium acetate:acetonitrile=65:35

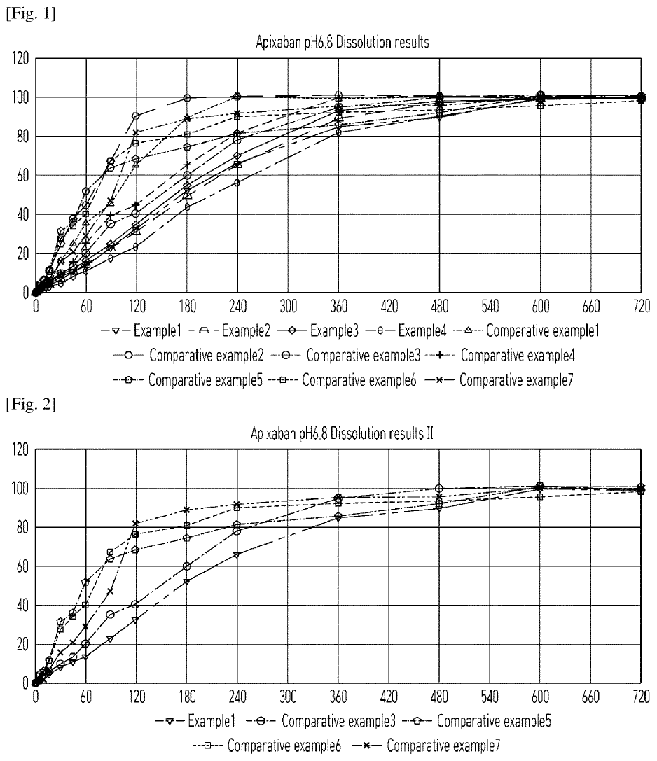

Results thereof are shown in FIGS. 1 and 2 and Table 2.

TABLE 2

| | | Example | Example | Example | Example | Comparative | Comparative |
|---|---|---|---|---|---|---|---|
| | | 1 | 2 | 3 | 4 | Example 1 | Example 2 |
| 15 | min | 3.8 | 3.5 | 4.4 | 3.1 | 4.5 | 10.2 |
| 30 | min | 8.9 | 6.8 | 9.9 | 4.7 | 16.6 | 25.4 |
| 60 | min | 13.4 | 14.5 | 16.6 | 11.1 | 35.5 | 45.3 |
| 180 | min | 52.1 | 49.1 | 55.1 | 44.1 | 89.4 | 99.9 |
| 360 | min | 85.0 | 89.0 | 92.7 | 82.2 | 99.1 | 101.2 |
| 600 | min | 99.4 | 99.9 | 100.4 | 99.9 | 100.4 | 100.0 |

| | | Comparative | Comparative | Comparative | Comparative | Comparative |
|---|---|---|---|---|---|---|
| | | Example 3 | Example 4 | Example 5 | Example 6 | Example 7 |
| 15 | min | 5.3 | 5.7 | 8.9 | 11.7 | 7.0 |
| 30 | min | 10.2 | 9.5 | 31.7 | 27.6 | 15.8 |
| 60 | min | 20.4 | 25.4 | 51.9 | 40.3 | 29.4 |
| 180 | min | 60.1 | 65.4 | 74.6 | 81.2 | 89.0 |
| 360 | min | 94.5 | 99.7 | 85.8 | 92.1 | 95.6 |
| 600 | min | 101.0 | 100.6 | 101.2 | 95.7 | 100.1 |

As seen in FIGS. 1, 2, and Table 2, the tablets of Examples 1 to 4 showed a dissolution rate of 20% or less of apixaban at 60 minutes from the start of dissolution, 40 to 60% at 180 minutes from the start of dissolution, and 80% or more at 360 minutes from the start of dissolution, confirming that the tablets of Examples had dissolution rates desirable for the once-daily apixaban formulation. On the other hand, the tablets of Comparative Examples 1 to 7 all showed a dissolution rate exceeding 20% at 60 minutes from the start of dissolution and exceeding 60% at 180 minutes from the start of dissolution, indicating that the tablets of Comparative Examples failed to show sustained release patterns desirable for the once-daily apixaban formulation.

### Experimental Example 2: Pharmacokinetics (PK) Test on Beagle Dogs

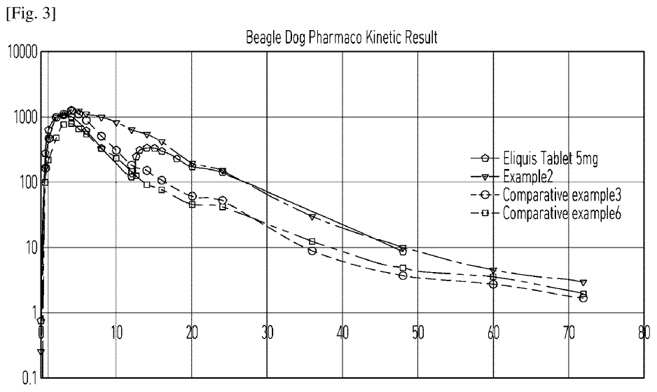

A pharmacokinetic (PK) test was performed using Beagle dogs with the tablets of Example 2, Comparative Examples 3 and 6, and Eliquis 5 mg tablets commercially available as a reference drug. The tablets of Example 2 and the preparations of Comparative Examples 3 and 6 were administered as a single dose due to their once-daily formulation. In contrast, the reference drug were administered an additional dose after 12 hours since it is formulated twice a day. The PK test results thereof are shown in FIG. 3 and Table 3.

TABLE 3

| | Reference drug | | Comparative | Comparative |
|---|---|---|---|---|
| | (Eliquis Tab) | Example 2 | Example 3 | Example 6 |
| AUCₗₐₛₜ (ng · | 11265.06 ± | 12030.43 ± | 7840.82 ± | 6789.88 ± |
| hr/mL) | 2829.81 | 3069.84 | 3118.21 | 6119.77 |
| Cₘₐₓ (hr) | 1065.08 ± | 1324.10 ± | 1266.51 ± | 803.04 ± |
| | 350.4 | 319.08 | 419.29 | 102.30 |
| Tₘₐₓ (hr) | 3.0 | 4.0 | 4.0 | 4.0 |
| | [1.0-4.0] | [2.0-5.0] | [2.0-5.0] | [2.0-5.0] |

As a result, as evident from FIG. 3 and Table 3, the tablets in Example 2 exhibited equivalent or similar levels of Cₘₐₓ and AUC compared to those of the reference drug. However, the AUCs for tablets in Comparative Examples 3 and 6 decreased significantly compared to AUC of the reference drug. In other words, Comparative Example 3 not containing TPGS as a surfactant or Comparative Example 6 containing a different type of surfactant instead of TPGS, failed to achieve equivalent or similar AUC values compared to those of the reference drug due to a significant decrease in the blood concentration of apixaban, the active ingredient. On the other hand, the tablet of the present disclosure containing TPGS as a surfactant (Example 2) ensured an equivalent or similar level of apixaban blood concentration and AUC values compared to the reference drug, and thus, it was confirmed that the tablet of the present disclosure was optimized as a once-daily sustained-release dosage form containing apixaban.

1. A sustained-release tablet comprising apixaban or a pharmaceutically acceptable salt thereof, polyethylene oxide as a sustained-release base and tocopherol polyethylene glycol succinate.

2. The sustained-release tablet of claim 1, wherein the polyethylene oxide is contained in an amount of 30% by weight to 60% by weight based on the total weight of a uncoated tablet.

3. The sustained-release tablet of claim 1, wherein the polyethylene oxide is contained in an amount of 37.5% by weight to 60% by weight based on the total weight of a uncoated tablet.

4. The sustained-release tablet of claim 1, wherein the polyethylene oxide is contained in an amount of 37.5% by weight to 45% by weight based on the total weight of a uncoated tablet.

5. The sustained-release tablet of claim 1, wherein the polyethylene oxide is at least any one selected from the group consisting of polyethylene oxide 300,000 and polyethylene oxide 1,000,000.

6. The sustained-release tablet of claim 4, wherein the sum of weights of polyethylene oxide 300,000 and polyethylene oxide 1,000,000 is 40% by weight to 60% by weight based on the total weight of the uncoated tablet.

7. The sustained-release tablet of claim 1, wherein the tocopherol polyethylene glycol succinate is contained in an amount of 0.25% by weight to 2.5% by weight based on the total weight of a uncoated tablet.

8. The sustained-release tablet of claim 1, wherein the tocopherol polyethylene glycol succinate is contained in 0.75% by weight to 2.5% by weight based on the total weight of a uncoated tablet.

9. The sustained-release tablet of claim 1, wherein the tocopherol polyethylene glycol succinate is contained in an amount of 0.75% by weight to 1.25% by weight based on the total weight of a uncoated tablet.

10. The sustained-release tablet of claim 1, further comprising at least any one selected from the group consisting of an excipient, a solubilizer, an antioxidant, a binder, a lubricant and a coating agent.

11. The sustained-release tablet of claim 10,
wherein the apixaban is contained in an amount of 2 to 3% by weight based on the total weight of a uncoated tablet,
the excipient is contained in an amount of 30 to 55% by weight based on the total weight of the uncoated tablet,
the solubilizer is contained in an amount of 0.1 to 2% by weight based on the total weight of the uncoated tablet,
the antioxidant is contained in an amount of 0.1% to 1% by weight based on the total weight of the uncoated tablet,
the binder is contained in an amount of 0.1 to 2% by weight based on the total weight of the uncoated tablet, and
the lubricant is contained in an amount of 0.1 to 2% by weight based on the total weight of the uncoated tablet.

12. The sustained-release tablet of claim 1, wherein the sustained-release tablet shows a dissolution rate of apixaban of 20% or less at 60 minutes, 40 to 60% at 180 minutes, and 80% or more at 360 minutes in a dissolution test with a pH 6.8 buffer solution at 100 rpm according to the dissolution test method 2 of the Korean Pharmacopoeia General Test Method.

13. The sustained-release tablet of claim 1, wherein the sustained-release tablet is for once-daily administration.
